(19) United States
(12) Patent Application Publication
(10) Pub. No.: US 2009/0088828 A1
(43) Pub. Date: Apr. 2, 2009

(54) Electrically Charged Implantable Medical Device

(75) Inventors: Alon Shalev, RaAnana (IL); Alexander Dubson, Petach-Tikva (IL)

Correspondence Address: MARTIN D. MOYNIHAN d/b/a PRTSI, INC., P.O. BOX 16446, ARLINGTON, VA, 22215, US

(73) Assignee: Nicast Ltd., Lod (IL)

(21) Appl. No.: 11/920,416

(22) PCT Filed: May 17, 2006
(86) PCT No.: PCT/IL2006/000584

§ 371 Date: Nov. 15, 2007

Related U.S. Application Data
(60) Provisional application No. 60/681,468, filed on May 17, 2005.

(51) Int. Cl. A61F 2/82

(52) U.S. Cl. 623/1.2

(57) ABSTRACT

A medical device comprises a tubular structure adapted for being implanted in the vasculature of a mammal, the tubular structure being formed, at least in part, of electrically charged nonwoven polymer fibers.

### FIELD AND BACKGROUND OF THE INVENTION

The present invention relates to medical devices, and, more particularly, to an implantable medical device having electrostatic charge.

Coronary heart disease may result in stenosis, which results in the narrowing or constriction of an artery. Percutaneous coronary intervention (PCI) including balloon angioplasty and stent deployment is currently a mainstay in the treatment of coronary heart disease. This treatment is often associated with acute complications such as late restenosis of angioplastied coronary lesions.

Restenosis refers to the reclosure of a previously stenosed and subsequently dilated peripheral or coronary blood vessel. Numerous studies have allowed a better understanding of the biology of restenosis. Neointimal thickening, also referred to as neointimal hyperplasia, occurs in response to experimental arterial injury. This process involves different steps, which include smooth muscle cell activation, proliferation and migration, and the production of extracellular matrix. Neointimal thickening has been identified as one of the mechanisms of restenosis after balloon angioplasty in humans. The factors controlling neointimal thickening include growth factors, hormonal factors and mechanical factors.

In addition to neointimal thickening, arterial remodeling also plays a major role in restenosis. Studies performed on animals and in human subjects have established the potential for “constrictive remodeling” to reduce the vessel lumen after angioplasty. Restenosis therefore appears as a multifactorial entity that may be addressed in the future by a combined mechanical and pharmacological approach.

A common solution to restenosis is intercoronary stenting, which is intended to provide the coronary with radial support and thereby prevent constriction. A stent is transported by a balloon catheter to the defective site in the artery and then expanded radially by the balloon to dilate the site and thereby enlarge the passage through the artery. Clinical data indicates, however, that stents are usually unable to prevent late restenosis beginning at about three months following an angioplasty procedure.

Beside restenosis, PCI involves the risk of vessel damage during stent implantation. As the balloon and/or stent expands it then cracks the plaques on the wall of the artery and produces shards or fragments whose sharp edges cut into the tissue. This causes internal bleeding and a possible local infection, which if not adequately treated, may spread and adversely affect other parts of the body. The stent-induced mechanical arterial injury and a foreign-body response to the implanted stent are believed to result in acute and chronic inflammation in the vessel wall, leading to production of cytokines and growth factors. These are believed to activate multiple signaling pathways, inducing vascular smooth muscle cell proliferation, which, as stated, results in neointimal hyperplasia.

To date, attempts have been made to prevent neointimal formation and restenosis by systemic administration of drugs, and sometimes by intravascular irradiation of the angioplastied artery, however these attempts have not been successful. Hence, current research is being shifted gradually to the local administration of various pharmaceutical agents at the site of an arterial injury resulting from angioplasty. The advantages gained by local therapy include higher concentrations of the drug at the actual injury site. One example of such treatment is local drug delivery of toxic drugs such as taxol and rapamycin to the vessel site via a catheter-based delivery system. However, local treatment systems dispensing a medication on a one-shot basis cannot efficiently prevent late restenosis.

Numerous attempts to develop stents with a local drug-distribution function have been made, most of which are variances of the so called stent graft, a metal stent covered with polymer envelope, containing anti-coagulant and/or anti-proliferative medicaments. The therapeutic action of stent grafts is based on gradual decomposition of biodegradable polymers under the effect of aggressive biological medium and drug liberation into the tissues which is in direct contact with the stent graft location. Drug-loaded polymer can be applied by spraying or by dipping the stent graft into a solution or melt, as disclosed, for example, in U.S. Pat. Nos. 5,383,922, 5,824,048, 5,624,411 and 5,733,327. Additional method for providing a drug-loaded polymer is disclosed in U.S. Pat. Nos. 5,637,113 and 5,766,710, where a pre-fabricated film is attached to the stent. Other methods, such as deposition via photo polymerization, plasma polymerization and the like, are also known in the art and are described in, e.g., U.S. Pat. Nos. 3,525,745, 5,609,629 and 5,824,049.

Stents with fiber polymer coating promote preparation of porous coatings with better grafting and highly developed surface. U.S. Pat. No. 5,549,663 discloses a stent graft having a coating made of polyurethane fibers which are applied using conventional wet spinning techniques. Prior to the covering process, a medication is introduced into the polymer.

A more promising method for stent coating is electrospinning. Electrospinning is a method for the manufacture of ultra-thin synthetic fibers which reduces the number of technological operations required in the manufacturing process and improves the product being manufactured in more than one way. The use of electrospinning for stent coating permits to obtain durable coating with wide range of fiber thickness (from tens of nanometers to tens of micrometers), achieves exceptional homogeneity, smoothness and desired porosity distribution along the coating thickness. Stents themselves do not encourage normal cellular invasion and therefore can lead to an undisciplined development of cells in the metal mesh of the stent, giving rise to cellular hyperplasia. When a stent is electrospinningly coated by a graft of a porous structure, the pores of the graft component are invaded by cellular tissues from the region of the artery surrounding the stent graft. Moreover, diversified polymers with various biochemical and physico-mechanical properties can be used in stent coating. Examples of electrospinning methods in stent graft manufacturing are found in U.S. Pat. Nos. 5,639,278, 5,723,004, 5,948,018, 5,632,772 and 5,855,598.

In is known that the electrospinning technique is rather sensitive to the changes in the electrophysical and rheological parameters of the solution being used in the coating process. In addition, incorporation of drugs into the polymer in a sufficient concentration, so as to achieve a therapeutic effect, reduces the efficiency of the electrospinning process. Still in addition, drug introduction into a polymer reduces the mechanical properties of the resulting coat. Although this drawback is somewhat negligible in relatively thick films, for submicron fibers made film this effect may be adverse.

There is thus a widely recognized need for, and it would be highly advantageous to have, implantable vascular prosthesis and/or stent assembly devoid of the above limitations.

### SUMMARY OF THE INVENTION

According to one aspect of the present invention there is provided a medical device, comprising a tubular structure adapted for being implanted in the vasculature of a mammal, the tubular structure being formed, at least in part, of electrically charged nonwoven polymer fibers.

According to another aspect of the present invention there is provided a method of connecting a pair of blood vessels, comprising, providing the medical device, forming a pair of holes in the pair of blood vessels, and connecting the medical device to the pair of holes so as to allow blood flow through the medical device, thereby connecting the pair of blood vessels.

According to yet another aspect of the present invention there is provided a method of bypassing an obstructed portion of a blood vessel, comprising, providing the medical device, forming a pair of holes in the blood vessel upstream and downstream the obstruction, and connecting the medical device to the pair of holes so as to allow blood flow through the medical device.

According to still another aspect of the present invention there is provided a method of producing a medical device, comprising electrospinning at least one liquefied polymer onto a precipitation electrode such as to provide a tubular structure formed of electrically charged nonwoven polymer fibers.

According to further features in preferred embodiments of the invention described below, the precipitation electrode comprises a rotating mandrel.

According to still further features in the described preferred embodiments the precipitation electrode comprises an expandable tubular supporting element.

According to still further features in the described preferred embodiments the precipitation electrode comprises an expandable tubular supporting element mounted on a rotating mandrel.

According to still further features in the described preferred embodiments the method further comprises supplementing the liquefied polymer with a charge control agent, prior to the electrospinning, the charge control agent being selected such that the nonwoven polymer fibers maintain a sufficiently amount of electrical charge for at least T hours.

According to still further features in the described preferred embodiments the device further comprises an expandable tubular supporting element.

According to still further features in the described preferred embodiments the expandable tubular supporting element is coated by the tubular structure.

According to still further features in the described preferred embodiments the tubular structure serves as a liner for the expandable tubular supporting element.

According to still further features in the described preferred embodiments the expandable tubular supporting element is embedded within the tubular structure.

According to an additional aspect of the present invention there is provided a method of treating a constricted blood vessel, the method comprising placing the medical device in the constricted blood vessel.

According to further features in preferred embodiments of the invention described below, the method further comprises expanding the expandable tubular supporting element and the tubular structure so as to dilate tissues surrounding the device in a manner such that flow constriction is substantially eradicated.

According to still further features in the described preferred embodiments the nonwoven polymer fibers comprise electrospun polymer fibers.

According to still further features in the described preferred embodiments the tubular structure comprises at least a first layer having a predetermined first porosity and a second layer having a predetermined second porosity.

According to still further features in the described preferred embodiments the first layer is formed of a first type of nonwoven polymer fibers and the second layer is formed of a second type of nonwoven polymer fibers.

According to still further features in the described preferred embodiments the device further comprises a secondary tubular structure of nonwoven polymer fibers, the tubular structure and the secondary tubular structure being in fluid communication via an anastomosis such that the tubular structure terminates at the anastomosis and the secondary tubular structure continues at the anastomosis.

According to still further features in the described preferred embodiments the tubular structure comprises at least one part positively charged part and at least one negatively charged part.

According to still further features in the described preferred embodiments the tubular structure has a substantially zero overall net electrical charge.

According to still further features in the described preferred embodiments the tubular structure has an overall net positive electrical charge of at least 0.001 μC per gram in magnitude.

According to still further features in the described preferred embodiments the tubular structure has an overall net negative electrical charge of at least 0.001 μC per gram in magnitude.

According to still further features in the described preferred embodiments the electrically charged nonwoven polymer fibers are capable of discharging at least 90% of the electric charge carried thereby over a predetermined time interval.

According to still further features in the described preferred embodiments the predetermined time interval is defined from the implantation of the device in the vasculature to about 1 hour following the implantation.

According to still further features in the described preferred embodiments the predetermined time interval is defined from the implantation of the device in the vasculature to about 12 hours following the implantation.

According to still further features in the described preferred embodiments the predetermined time interval is defined from the implantation of the device in the vasculature to about 24 hours following the implantation.

According to still further features in the described preferred embodiments the predetermined time interval is defined from the implantation of the device in the vasculature to about 3 days following the implantation.

According to still further features in the described preferred embodiments the predetermined time interval is defined from about 3 days following the implantation of the device in the vasculature to about 7 days following the implantation.

According to still further features in the described preferred embodiments the predetermined time interval is defined from about 7 days following the implantation of the device in the vasculature to about 30 days following the implantation.

According to still further features in the described preferred embodiments the electrically charged nonwoven polymer fibers are capable of maintaining at least 90% of the electric charge carried thereby over a predetermined period following implantation of the device in the vasculature.

According to still further features in the described preferred embodiments the predetermined period equals about 3 days.

According to still further features in the described preferred embodiments the predetermined period equals about 7 days.

According to still further features in the described preferred embodiments the predetermined period equals about 30 days.

According to still further features in the described preferred embodiments the tubular structure comprises at least one pharmaceutical agent incorporated therein for delivery of the at least one pharmaceutical agent into the vasculature during or after implantation of the medical device within the vasculature.

The present invention successfully addresses the shortcomings of the presently known configurations by providing an electrically charged implantable medical device.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawing. With specific reference now to the drawing in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawing making apparent to those skilled in the art how the several forms of the invention may be embodied in practice.

In the drawings:

FIG. 1 is a schematic illustration of a medical device serving as a stent assembly, according to various exemplary embodiments of the present invention;

FIG. 2a is an end view the stent assembly, according to a preferred embodiment of the present invention;

FIG. 2b is an end view of a stent assembly which further comprises at least one adhesion layer, according to a preferred embodiment of the present invention;

FIG. 3 is a schematic illustration of tubular supporting element designed and constructed for dilating a constricted blood vessel in the body vasculature, according to various exemplary embodiments of the present invention;

FIG. 4 is a schematic illustration of a portion of the tubular supporting element of FIG. 3, comprising a deformable mesh of metal wires, according to a preferred embodiment of the present invention;

FIG. 5 is schematic illustration of a stent assembly, manufactured according to the teachings of the present invention, occupying a defective site in an artery;

FIG. 6 is schematic illustration of a portion of a non-woven web of polymer fibers produced according to various exemplary embodiments of the present invention;

FIG. 7 is schematic illustration of a portion of a non-woven web of polymer fibers which comprises a pharmaceutical agent constituted by compact objects and distributed between the electrospun polymer fibers;

FIGS. 8a-d are schematic illustrations of the medical device serving as a vascular prosthesis, according to various exemplary embodiments of the present invention;

FIG. 9 is a schematic illustration of the medical device in a preferred embodiment in which the medical device serves as a multiport vascular prosthesis;

FIGS. 10a-b which are schematic planar views of the multiport vascular prosthesis, according to various exemplary embodiments of the present invention;

FIG. 11 is a flowchart of a method suitable for producing the medical device, according to various exemplary embodiments of the present invention;

FIG. 12 is a schematic illustration of a system for manufacturing an electrospun tubular structure, according to various exemplary embodiments of the present invention; and

FIGS. 13a-b are schematic illustrations of an apparatus for manufacturing a multiport electrospun structure, according to various exemplary embodiments of the present invention.

### DESCRIPTION OF THE PREFERRED EMBODIMENTS

The present embodiments comprise a medical device which can be used as a stent or a vascular prosthesis. Specifically, the present embodiments can be used for treating a constricted blood vessel, forming a bypass in the vasculature (e.g., to bypass an obstructed blood vessel), connecting blood vessels, and the like. The present embodiments further comprise a method for manufacturing the medical device and various applications using the medical device.

The principles and operation of a method and device according to the present embodiments may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In a search for a technique to reduce or prevent neointimal proliferation and subsequent restenosis, the present Inventors have unexpectedly uncovered that an electrically charged implant can be used as neointimal proliferation inhibitor. Unlike systemic administration of anti-proliferative drugs, the technique of the present embodiment offers the option of local treatment, in a similar manner to the treatment performed, e.g., by drug eluting stents.

According to one aspect of the present invention there is provided a medical device which comprises a tubular structure formed, at least in part, of electrically charged nonwoven polymer fibers. The tubular structure is preferably adapted for being implanted in the vasculature of a mammal. Preferred internal diameter of the tubular structure is from about 1 mm to about 30 mm, more preferably from about 2 mm to about 20 mm, most preferably from about 2 mm to about 6 mm. Preferred wall thickness for the tubular structure is in the range between about 0.01 mm to about 1 mm, more preferably, between about 0.1 mm to about 0.5 mm, inclusively.

As used herein the term “about” refers to ±10%.

The electrical charge or, more specifically, electrostatic charge carried by the nonwoven polymer fibers provides protection from thrombosis due to its ability to prevent or significantly reduce cell proliferation and inflammatory reaction. The interaction between the electrical charge and one or more biological substances present in the vasculature, directly or indirectly results in generation of oxidizing agents in amounts sufficient to prevent cell proliferation. The generation of oxidizing agents is from body constituents, such as, chlorine ions, molecular oxygen and water which are normally present in sufficient amounts in the blood.

According to a preferred embodiment of the present invention the electrically charged nonwoven polymer fibers carry sufficient amount of electrical charge for preventing or reducing neointimal proliferation and/or formation of thrombus.

As used herein, the phrase “preventing or reducing neointimal proliferation” is equivalent to “preventing or reducing cell growth” and includes killing cells, inducing tissue necrosis, inducing cell apoptosis and/or inducing cell growth arrest.

According to a preferred embodiment of the present invention the tubular structure comprises differently charged parts. For example, one or several parts of the tubular structure can be positively charged part and one or several parts can be negatively charged. Alternatively or additionally, different parts of the tubular structure can have charge of the same polarity but different magnitude.

In various exemplary embodiments of the invention the tubular structure has a substantially zero overall net electrical charge. In other embodiments the overall net electrical charge of the tubular structure (in absolute value) is larger than 0.001 μC per gram, more preferably larger than 0.01 μC per gram more preferably 0.1 μC per gram.

The electrically charged nonwoven polymer fibers are preferably capable of discharging 90% or more of the electric charge carried thereby over a predetermined time interval, which can be defined, for example, from the implantation of the device in the vasculature to about 1 hour, 12 hours, 24 hours or several days (e.g., 3 days) following implantation.

In another embodiment, the nonwoven polymer fibers maintain the electrical charge for a few, say 3, 7, 10, or 14 days. In this embodiment the polymer fibers preferably discharge the electrical charge over a predetermined time interval which begins several days after the implantation of the device. For example, the polymer fibers can discharge 90% or more of the electric charge over a time interval, which begins about 3 days after implantation and ends about 7 days after implantation. Also contemplated is a medical device in which the polymer fibers discharge 90% or more of the electric charge over a time interval, which begins about 7 days after implantation and ends about 30 days after implantation.

The time interval during which the electrically charged nonwoven polymer fibers discharge, and the duration for which they maintain the electrical charge can be selected during the manufacturing process of the tubular structure. In various exemplary embodiments of the invention the bulk electrical properties (e.g., electrical conductivity, electrical resistivity, permittivity, dielectric constant) of the polymer used for forming the fibers are selected such that a sufficiently amount of electrical charge is maintained in the fibers for at least T hours, where T is from about 1 hour to about 3 months. The bulk electrical properties are also selected to enable the desired discharge time interval.

The discharge time interval also depends on the surface properties of the fibers. For example, upon implantation of the medical device in an aqueous medium (e.g., blood vessel), fibers having hydrophobic properties tend to be discharged at a lower rate than fibers having hydrophilic properties. Also, fibers of higher electrical resistivity on their surface tend to tend to be discharged at a lower rate than fibers of low electrical resistivity. Thus, according to a preferred embodiment of the present invention the bulk properties and/or the surface properties are selected such as to enable the desired discharge time interval and/or the desired period over which the electrical charge is maintained. The bulk properties and/or the surface properties may include any attribute of the polymer from which the electrically charged nonwoven fibers are formed, including, without limitation, hydrophobic properties and electrostatic properties. The desired properties can be achieved by a judicious selection of the polymer used and/or by supplementing the polymer with additives. For example, to enhance the hydrophobic properties of the polymer fibers, the polymer can be supplemented with of a siloxane.

Preferably, a larger portion of the electrical charge is maintained in the bulk of the fibers while smaller portion thereof is a surface charge. More preferably, but not obligatorily, all the electrical charge is maintained in the bulk of the fibers.

The advantage of discharging during a relatively short interval (e.g., within 1 or several hours post implantation) is the ability to address acute post-surgical effects, such as immediate tissue injury. The advantage of discharging during relatively long intervals (e.g., within several hours or several days to several months post implantation) is the ability to address longer-term processes, such as smooth muscle cell migration and proliferation.

Typical thickness of the polymer fibers is, without limitation, from about 50 nm to about 5000 nm, more preferably from about 100 nm to about 500 nm.

The polymer fibers can be manufactured using any technique for forming nonwoven fibers, such as, but not limited to, an electrospinning technique, a wet spinning technique, a dry spinning technique, a gel spinning technique, a dispersion spinning technique, a reaction spinning technique or a tack spinning technique.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference.

Other spinning techniques are disclosed, e.g., U.S. Pat. Nos. 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

A preferred technique for manufacturing a medical device suitable for the present embodiments is provided hereinunder.

Following is a description of several application for which the medical device of the present embodiments can be useful.

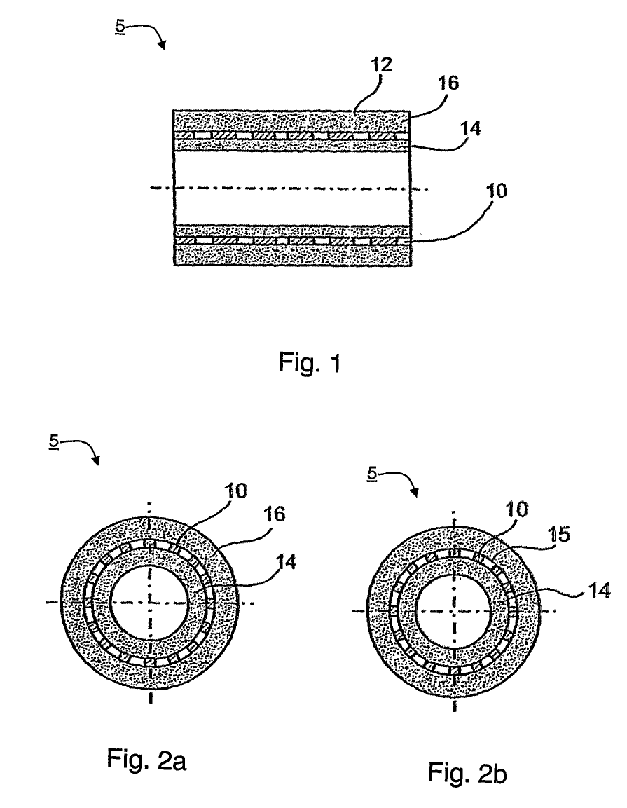

FIG. 1 is a schematic illustration of a medical device 5 in a preferred embodiment in which the medical device serves as a stent assembly. The stent assembly comprises an expendable tubular supporting element 10 and a tubular structure 12. Element 10 can be coated by or embedded in tubular structure 12. In the embodiment in which element 10 is coated by structure 12, it can be coated externally and/or internally by structure 12. In other words, structure 12 can serve as an outer coat (a cover layer), an inner coat (a liner layer) or both an outer coat and an inner coat. Show in FIG. 1 is a preferred embodiment in which structure 12 comprises an inner coat 14, lining an inner surface of element 10 and an outer coat 16, covering an outer surface of element 10.

FIG. 2a illustrates an end view of the stent assembly, showing element 10, internally covered by inner coat 14 and externally covered by outer coat 16. With reference to FIG. 2b, coat 12 may further comprise at least one adhesion layer 15, for adhering the components of the stent assembly as further detailed hereinafter.

According to a preferred embodiment of the present invention inner 14 and outer 16 coats are made of different polymer fibers and have predetermined porosities, which may be different or similar as desired.

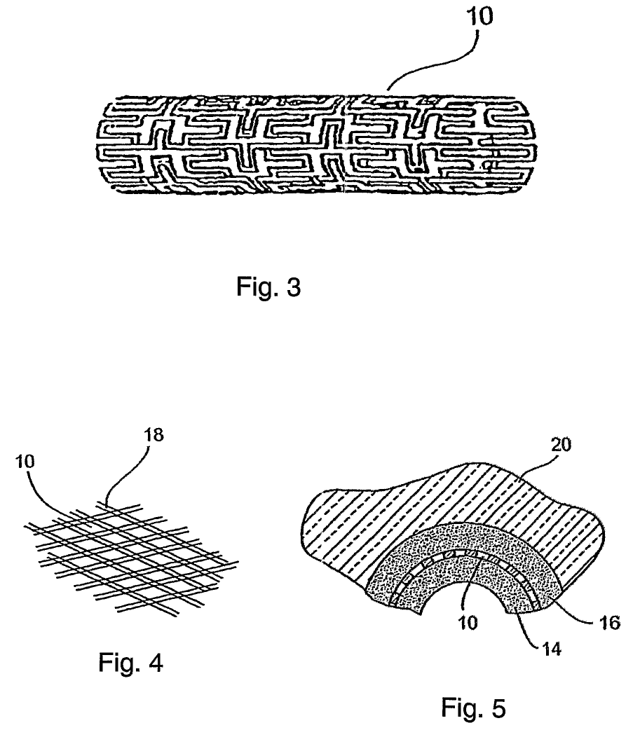

Reference is now made to FIG. 3 which is a schematic illustration of tubular supporting element 10 designed and constructed for dilating a constricted blood vessel in the body vasculature. Element 10 expands radially thereby dilates a constricted blood vessel. According to a preferred embodiment of the present invention, the expansibility of the stent assembly may be optimized by a suitable construction of element 10 and tubular structure 12. The construction of element 10 will be described first, with reference to FIG. 4, and the construction of structure 12 will be described thereafter.

Hence, FIG. 4 illustrates a portion of element 10 comprising a deformable mesh of metal wires 18, which can be, for example, a deformable mesh of stainless steel wires. When the stent assembly is placed in the desired location in an artery, element 10 may be expanded radially, to substantially dilate the arterial tissues surrounding the stent assembly to eradicate a flow constriction in the artery. The expansion may be performed by any method known in the art, for example by using a balloon catheter or by forming element 10 from a material exhibiting temperature-activated shape memory properties, such as Nitinol. According to a presently preferred embodiment of the invention, the polymer fibers forming structure 12 are elastomeric polymer fibers which stretch as element 10 is radially expanded. According to a preferred embodiment of the present invention inner coat 14 and outer coat 16 are coextensive with element 10, i.e., tubular supporting element 10 is substantially coated. Alternatively, inner coat 14 and/or outer coat 16 may be shorter in length than element 10, in which case at least one end of element 10 is exposed.

Reference is now made to FIG. 5, which illustrates the stent assembly occupying a defective site 20 in an artery. The outer diameter of the stent assembly in its unexpanded state, including outer coat 16, is such that it ensures transporting of the stent assembly through the artery to defective site 20, for example by a catheter. The expending range of the stent assembly is such that when in place at defective site 20, the expanded assembly then has a maximum diameter causing the arterial tissues surrounding the stent assembly to be dilated to a degree eradicating the flow constriction at the site.

In various exemplary embodiments of the invention structure 12 comprises a pharmaceutical agent incorporated therein for delivery of the pharmaceutical agent into the vasculature during or after the implantation of the medical device within the vasculature. Thus, in this embodiment, structure 12 serves not only as an electrical charge carrier as explained above, but also as a reservoir for storing the pharmaceutical agent to be delivered over a prolonged time period. The pharmaceutical agent can be a treating agent (e.g., a medicament or a drug), a diagnostic agent (e.g., an imaging agent), or any other pharmaceutical composition, i.e., a preparation of one or more active ingredients with other chemical components such as physiologically suitable carriers and excipients.

Representative examples of suitable pharmaceutical agents, include, without limitation, antithrombotic, estrogens, corticosteroids, cytotoxic, cytostatic, anti-coagulant, vasodilator, antiplatelet, thrombolytics, antimicrobials, antibiotics, antimitotics, antiproliferatives, antisecretory, non-steroidal antiflammentory, growth factor antagonists, free radical scavengers, antioxidants, radiopaque agents, nitric-oxide donors, immunosuppressants and radio-labeled agents.

Representative examples of suitable drugs include, without limitation, heparin, tridodecylmethylammonium-heparin, epothilone A, epothilone B, sirolimus, tacrolimus, cyclosporine, rotomycine, ticlopidine, dexamethasone and caumadin.

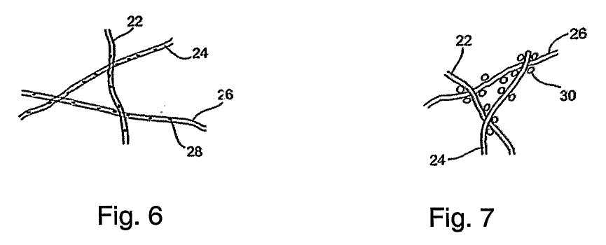

Reference is now made to FIG. 6 which illustrates a portion of a nonwoven web of polymer fibers according to a preferred embodiment of the present invention. Fibers 22, 24 and 26 intersect and are joined together at the intersections, the resultant interstices rendering the web highly porous. Since the fibers are ultra-thin, they have an exceptionally large surface area, which allows a high quantity of pharmaceutical agents to be incorporated thereon. Within the above fiber thickness limitations, the surface area of the fibers approaches that of activated carbon, thereby making the nonwoven web of polymer fibers an efficient local drug delivery system.

The preferred mechanism of pharmaceutical agent release from the fibers is by diffusion, regardless of the technique employed to embed the pharmaceutical agent therein. The duration of therapeutic drug release in a predetermined concentration depends on several variants, which may be controlled during the manufacturing process. One variant is the chemical nature of the carrier polymer and the chemical means binding the pharmaceutical agent to it. This variant may be controlled by a suitable choice of the polymer(s) used in the manufacturing process, which, in this embodiment, is preferably electrospinning. Another variant is the area of contact between the body and the pharmaceutical agent, which can be controlled by varying the free surface of the electrospun polymer fibers. Also affecting the duration of pharmaceutical agent release is the method used to incorporate the pharmaceutical agent within structure 12, as is further described hereinbelow.

According to a preferred embodiment of the present invention, the tubular structure comprises a number of sub-layers. Depending on their destination, the sub-layers can be differentiated by fiber orientation, polymer type, pharmaceutical agent incorporated therein and desired release rate thereof. Thus, pharmaceutical agent release during the first hours and days following implantation may be achieved by incorporating a solid solution, containing a medicament such as anticoagulants and antithrombogenic agents, in a sub-layer of readily soluble biodegradable polymer fibers. During the first period following implantation the medicament that releases includes anticoagulants and antithrombogenic agents.

Referring now again to FIG. 6, the pharmaceutical agent may be constituted by particles 28 embedded in the electrospun polymer fibers forming a sub-layer of tubular structure 12. This method is useful for pharmaceutical agent release during the first post-operative days and weeks. To this end, the pharmaceutical agent can include antimicrobials or antibiotics, thrombolytics, vasodilators, and the like. The duration of the delivery process is effected by the type of polymer used for fabricating the corresponding sub-layer. Specifically, optimal release rate is ensured by using moderately stable biodegradable polymers.

Reference is now made to FIG. 7 which schematically illustrates an alternative method for incorporating the pharmaceutical agent in the tubular structure, ensuring pharmaceutical agent release during the first post-operative days and weeks. Thus, according to a preferred embodiment of the present invention, the pharmaceutical agent is constituted by compact objects 30 distributed between the electrospun polymer fibers of the tubular structure. Compact objects 30 may be in any known form, such as, but not limited to, moderately stable biodegradable polymer capsules.

The present invention is also provides a method of releasing pharmaceutical agent, which may last from several months to several years. According to a preferred embodiment of the present invention the pharmaceutical agent is dissolved or encapsulated in a sub-layer made of biostable fibers. The rate diffusion from within a biostable sub-layer is substantially slower, thereby ensuring a prolonged effect of pharmaceutical agent release. Pharmaceutical agents suitable for such prolonged release include medicaments, such as, but not limited to, antiplatelets, growth-factor antagonists and free radical scavengers.

Thus, the sequence of pharmaceutical agent release and impact longevity of a certain specific pharmaceutical agents is determined by the type of polymer, the method in which the pharmaceutical agent is introduced into the polymer fibers, the sequence of layers forming the tubular structure, the matrix morphological peculiarities of each layer and the concentration of the pharmaceutical agent.

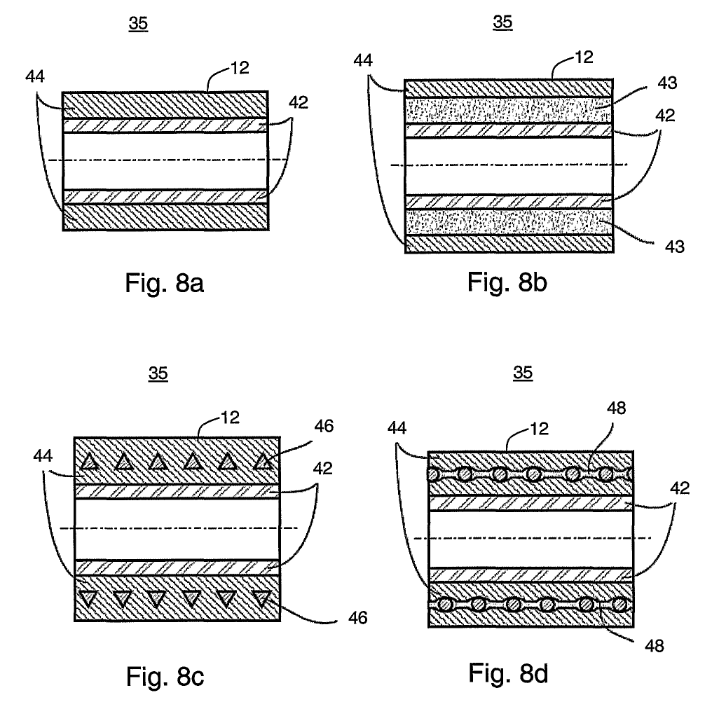

Reference is now made to FIGS. 8a-d which are schematic illustrations of a medical device 35 in a preferred embodiment in which the medical device serves as a vascular prosthesis. According to the presently preferred embodiment of the invention the tubular structure 12 has a first layer 42 characterized by a predetermined first porosity and a second layer 44 characterized a predetermined second porosity. First layer 42 and second layer 44 can be made of similar or different nonwoven polymer fibers, as desired. As shown in FIGS. 8a-d, first layer 42 is an inner layer (liner layer) and second layer 44 is an outer layer (cover layer) of the vascular prosthesis. Each of first 42 and second 44 layers can be made, at least in part, of electrically charged nonwoven fibers as further detailed hereinabove. According to a preferred embodiment of the present invention the inner layer is electrically charged so as to minimize thrombogenicity of the blood-contacting surface while the outer layer is charged substantially differently so as to minimize smooth muscle cell migration and proliferation.

First layer 42 is preferably manufactured substantially as a smooth surface with relatively low porosity. First layer 42 serves as a sealing layer to prevent bleeding, hence precludes preclotting, the rate of which is known to be high up to several hours after implantation. In addition, throughout the life of the vascular prosthesis, first layer 42 ensures antithrombogenic properties and efficient endothelization of the inner surface of the vascular prosthesis. A typical thickness of first layer 42 is ranging from about 40 μm to about 80 μm.

According to a preferred embodiment of the present invention second layer 44 provides requisite mechanical properties of the vascular prosthesis, specifically high compliance and high breaking strength, hence the thickness of second layer is preferably larger than the thickness of first layer 42. A typical thickness of second layer 44 is ranging from about 50 μm to about 1000 μm. In addition, the predetermined porosity of second layer 44 is preferably larger than the predetermined porosity of first layer 42. A porous structure is known to promote ingrowth of surrounding tissues, which is extremely important for fast integration and long-term patency of the vascular prosthesis.

According to a preferred embodiment of the present invention, the vascular prosthesis further comprises one or more layers 43 (shown in FIG. 8b), interposed between first layer 42 and second layer 44, each of the intermediate layers 43 is also made of nonwoven polymer fibers and having a predetermined porosity. The type of polymer can be similar or different from any of the types of polymers forming layers 42 and 44. Intermediate layer(s) 43 can also be electrically charged, if desired. In various exemplary embodiments of the invention the porosity level of the vascular prosthesis is a decreasing function of a distance from the center of the vascular prosthesis, but it should be understood that in other embodiments any predetermined porosity distributions may be employed. A multilayer vascular prosthesis can be used in cases of high bleeding hazard, for example, upon implantation of a shunt, which serves as a channel for fluid delivery in or out of the body vasculature.

Delivery or pharmaceutical agents into the body vasculature can be performed during or after implantation of the vascular prosthesis within the body vasculature. In this embodiment first layer 42 second layer 44 or any intermediate layers 43 may incorporate one or more pharmaceutical agents therein for delivery into body vasculature as further detailed hereinabove.

Reference is now made to FIG. 8c, which depicts a longitudinal cross-sectional view of the vascular prosthesis, in a preferred embodiment in which the vascular prosthesis is reinforced by one or more coiled patterns 46. The coiled patterns serve for reinforcing of the vascular prosthesis specifically for enhancing anti-kinking properties. Reinforced vascular prosthesis can be used, for example, upon implantation of long grafts within body vasculature, where the graft should fit the complex geometry of the host.

In accordance with the presently preferred embodiment of the invention, coiled pattern 46 is formed from a wound filament, which may be for example, a wound polypropylene filament or a wound polyurethane filament. The transverse cross section of the wound filament may be chosen so as to increase the mechanical properties of the vascular prosthesis.

As shown in FIG. 8c, the wound filament has a triangular cross section, however any other transverse cross sections may be selected, for example polygonal (other than a triangle) cross sections, a circular cross section, an ellipsoid cross section and an irregular pattern cross section.

Reference is now made to FIG. 8d, which depicts a longitudinal cross-sectional view of the vascular prosthesis, in a preferred embodiment in which the vascular prosthesis comprises a plurality of adhesion sublayers 48. The adhesion sublayers 48 can be alternately interposed between first layer 42 and coiled pattern 46, between coiled pattern 46 and second layer 44, and/or between two congruent coiled patterns (in cases where more than a single coiled pattern exists). Adhesion sublayers 48 serve for adhering the various layers to one another and may be either impervious or permeable.

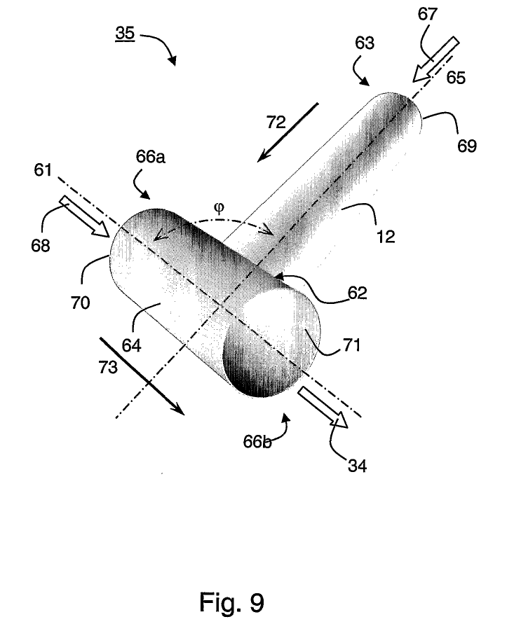

Reference is now made to FIG. 9 which is a schematic illustration of medical device 35 in a preferred embodiment in which the medical device serves as a multiport vascular prosthesis. According to a preferred embodiment of the present invention the vascular prosthesis comprises, in addition to structure 12, a secondary tubular structure 64. Structure 64 is preferably formed of nonwoven polymer fibers, and can include any number of layers as further explained above with respect to structure 12.

Tubular structures 12 and 64 are in fluid communication via an anastomosis 62, such that structure 12 terminates at anastomosis 62 while secondary structure 64 continues at anastomosis 62. Thus, structure 12 has one free end (designated on FIG. 9 by numeral 63), and structure 64 has two free ends (designated on FIG. 9 by numerals 66a and 66b). Anastomosis 62 is characterized by an anastomosis angle φ which is conveniently defined as the acute angle between the longitudinal axes of structures 12 and 64 (generally shown at 65 and 61). Preferred values for φ are from about 10 degrees to about 70 degrees, more preferably from about 20 degrees to about 50 degrees.

Preferred internal diameter of the tubular structures is from about 1 mm to about 30 mm, more preferably from about 2 mm to about 20 mm, most preferably from about 2 mm to about 6 mm. Preferred wall thickness for said tubular structures is in the range between about 0.1 mm to about 2 mm, more preferably, between about 0.5 mm to about 1.5 mm.

Typically, but not obligatorily, the length of tubular structure 12 is larger than the length of tubular structure 64. A preferred length of tubular structure 12 is from about 1 cm to about 70 cm, more preferably from about 15 cm to about 40 cm. A preferred length of tubular structure 64 is from about 10 mm to about 40 mm, more preferably from about 15 mm to about 35 mm.

In use, the multiport vascular prosthesis preferably receives arterial blood flow 67 from an artery (not shown) and vein blood flow 68 from a vein (not shown). An output blood flow 34 which includes a mixture of blood 68 and blood 67 is supplied to the vein. As shown in FIG. 9, arterial blood flow 67 enters through a primary input port 69, located at the free end 63 of structure 12, and vein blood flow 68 enters through a secondary input port 70, located at free end 66a of structure 64. The blood flows through the lumens of structures 12 and 64 and exits through an output port 71, located at free end 66b of structure 64. According to a preferred embodiment of the present invention the acute side anastomosis 62 faces secondary input port 70 and the obtuse side anastomosis 62 faces output port 71. Device 35 is therefore a multiport vascular prosthesis in the sense that it includes more than two ports (two input ports and one output port, in the present exemplary embodiment).

There is a preferred flow direction of blood through the multiport vascular prosthesis. In structure 12 the preferred flow direction is into anastomosis 62, and in secondary structure 64 the preferred flow direction is into output port 71. The preferred flow directions are illustrated in FIG. 9 by arrows 72 (flow in structure 12) and 73 (flow in structure 12). As will be appreciated by one of ordinary skill in the art the desired flow direction is enabled by the natural blood pressure in the vasculature and the choice of anastomosis angle φ and. In particular, high arterial blood pressure prevents blood from flowing upstream within structure 12. In turn, and the acute anastomosis angle φ at the side of port 70 and the blood pressure from the veins (although being lower than the arterial pressure), directs the blood flow to output port 71.

While reducing the present invention to practice it has be uncovered that a proper blood flow through the prosthesis can be ensured by a judicious construction of the profile of the prosthesis. In particular it was found that the shape of the profile of structure 12 can facilitate blood flow from structure 12 to secondary structure 64 (via anastomosis 62), such that the blood flows through structure 64 in direction 73.

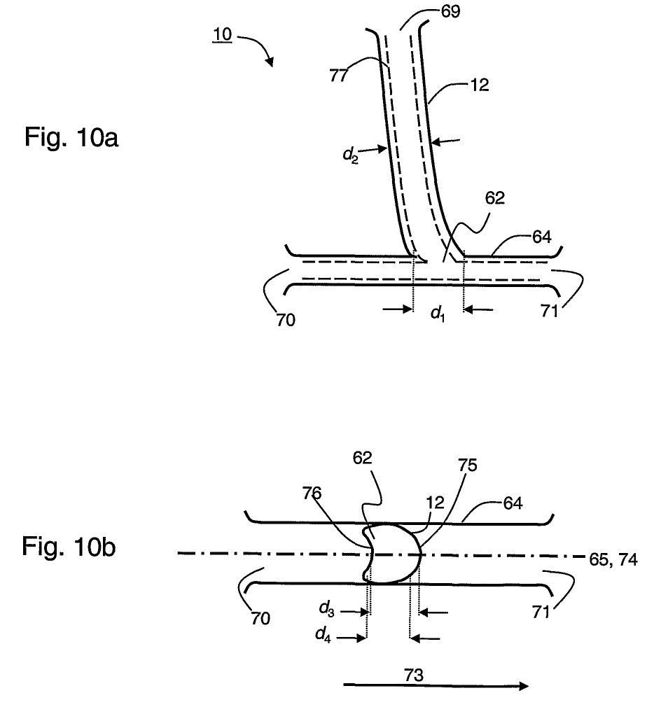

Reference is now made to FIGS. 10a-b, which are schematic planar views of the multiport vascular prosthesis, where FIG. 10a is a top view, illustrating the profiles (longitudinal cross sections) of tubular structures 12 and 64, and FIG. 10b is a side view illustrating the transverse cross section of structure 12 and the profile of structure 64, according to various exemplary embodiments of the present invention. One skilled in the art will recognize that several numerals have been omitted from FIGS. 10a-b for clarity of presentation.

According to a preferred embodiment of the present invention structure 12 widens towards anastomosis 62. In other words, the diameter defining structure 12 is larger at or near anastomosis 62 than far from anastomosis 62. For example, referring to FIG. 10a, the diameter d, at or near anastomosis 62 is larger than the diameter d₂ about half the distance between anastomosis 62 and port 69. Additionally, referring to FIG. 10b, the diameter of structure 12 at anastomosis 62 is larger at a plane 74 defined by longitudinal axes 65 and 61 than far from plane 74. The diameter at plane 74 is designated in FIG. 10b by d₃ and the diameter farther from plane 74 is designated by d₄.

According to a preferred embodiment of the present invention at least a part of structure 12 (preferably the part near or at anastomosis 62) is characterized by a cross section which is concave at one side of anastomosis 62. More preferably, but not obligatorily, at least a part of the cross section of structure 12 is concave at one side of anastomosis 62 and convex at the opposite side thereof.

As used herein, “concave” and “convex” describe the contour of the inner wall of the tubular structure.

In the representative example of FIG. 10b, the cross section of structure 12 is concave at the side facing output port 71 and convex at the side facing secondary input port 70. The concave and convex parts of the cross section are designated in FIG. 10b by numerals 75 and 76, respectively.

In various exemplary embodiments of the invention each one of ports 69, 70 and 71 is independently concave outwardly, to facilitate entrance/exit of blood flow to prosthesis 10 and to reduce risk of blood coagulation near the ends of the tubular structures.

Optionally and preferably, the multiport vascular prosthesis of the present embodiments can also comprise a support structure 77, extending from port 69 to anastomosis 62, and optionally also from port 70 to port 71. Support structure 77 can be disposed internally within the tubular structures, as illustrated in FIG. 10a, or it can be embedded in the walls of the tubular structures, as further detailed hereinbelow. Support structure 77 can be any support structure known in the art (to this end see, e.g., WO 02/49535 supra, and U.S. Pat. Nos. 6,945,993, 6,949,120 and 6,939,373). For example, structure 77 can be a deformable mesh of wires made of a metallic material such as, but not limited to, medical grade stainless steel or a material exhibiting temperature-activated shape memory properties, such as Nitinol.

As stated, each of structures 12 and 64 can include more than one layer of nonwoven polymer fibers. A particular additional advantage of the multilayer embodiment is that such configuration provides self-sealing properties to the multiport vascular prosthesis. This is particularly useful when the prosthesis is used as an arteriovenous shunt, in which case the prosthesis is punctured repeatedly. Self-sealing properties can be achieved by providing three or more layers, where the liner layer and the cover layer are formed from crude fibers with predominantly circumferential orientation, with relatively low porosity (say, from about 50% to about 70%), and the intermediate layer is formed from thin and randomly-oriented fibers, with relatively higher porosity (from about 80% to about 90%). Preferably, the intermediate layer comprises about 70% of the overall wall thickness of prosthesis 10. In accordance to the presently preferred embodiment of the invention, the inner layer and the outer layer, serve for supporting the intermediate layer.

Upon puncturing, a needle passes through the intermediate layer, by forcing the fibers apart, hence no rupturing occurs. The tearing is prevented due to the combination of high elasticity of the fibers, large number of voids and small number of bonds between the fibers. Once the needle extracted the original fibers web is reconstructed, both because of the fiber elasticity and because the pressure applied by the inner and outer layers. Thus, high level of sealing or re-annealing is achieved.

The strength properties of the prosthesis are mainly ensured by its inner and outer layers. The piercing damage in the outer and inner layers are spread apart of one another by a certain distance, thus minimizing the affect of puncture on the wall strength.

The vascular prosthesis of the present embodiments has numerous of physical, mechanical and biological properties. The properties of the vascular prosthesis can be any combination of the following characteristics (a) having an inner diameter expandable by at least 10% under a pulsatile pressure characterizing a mammalian blood system; (b) capable of maintaining said inner diameter while bent at a bent diameter of twice said inner diameter; (c) having a porosity of at least 60%; (d) preventing leakage of blood passing therethrough; (e) characterized by tissue ingrowth and cell endothelization over at least 90% of the vascular prosthesis within at least 10 days from implantation in a mammal; and (f) having a self-sealing properties so as to minimize blood leakage following piercing.

The combination of the vascular prosthesis mechanical characteristics, specifically high breaking strength, an admissible compliance level and porosity, stems from the electrospinning method of manufacturing, which is further described hereinunder.

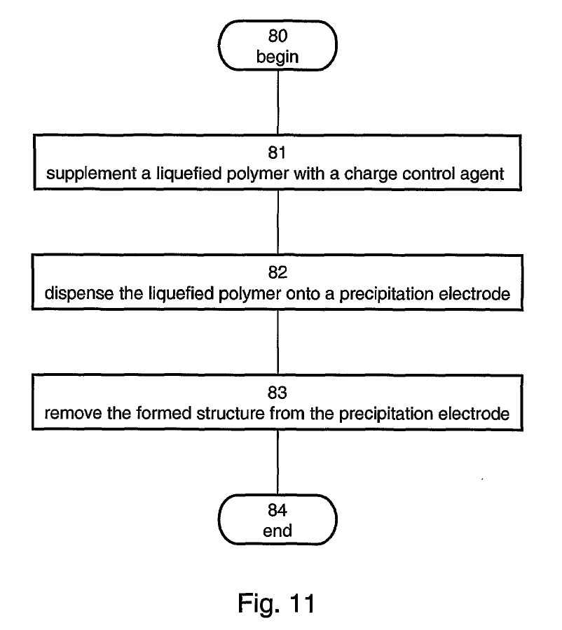

Reference is now made to FIG. 11 which is a flowchart of a method suitable for producing the medical device, according to various exemplary embodiments of the present invention.

It is to be understood that, unless otherwise defined, the method steps described hereinbelow can be executed either contemporaneously or sequentially in many combinations or orders of execution. Specifically, the ordering of the flowchart of FIG. 11 is not to be considered as limiting. For example, two or more method steps, appearing in the following description or in the flowchart of FIG. 11 in a particular order, can be executed in a different order (e.g., a reverse order) or substantially contemporaneously. Additionally, several method steps appearing in the following description or in the flowchart of FIG. 11 are optional and can be omitted.

The method described herein is based on the electrospinning process which is well-known to those skilled in the art of nonwoven polymer fibers. Hence, the method begins at step 80 and, optionally and preferably, continues to step 81 in which a liquefied polymer suitable for forming polymer fibers via electrospinning is supplemented (e.g., mixed) with a charge control agent (e.g., a dipolar additive). The liquefied polymer and the supplemented charge control agent can form, for example, a polymer-dipolar additive complex which apparently better interacts with ionized air molecules compared to the liquefied polymer per se. The charge control agent is preferably selected such that nonwoven polymer fibers produced during electrospinning process maintain a sufficiently amount of electrical charge for at least T hours, where T is from about 1 hour to about 3 months.

The charge control agent is typically added in the grams equivalent per liter range, say, in the range of from about 0.001 N to about 0.1 N, depending on the respective molecular weights of the polymer and the charge control agent used.

U.S. Pat. Nos. 5,726,107; 5,554,722; and 5,558,809 teach the use of charge control agents in combination with polycondensation processes in the production of electret fibers, which are fibers characterized in a permanent electric charge, using melt spinning and other processes devoid of the use of a precipitation electrode. A charge control agent is added in such a way that it is incorporated into the melted or partially melted fibers and remains incorporated therein to provide the fibers with electrostatic charge which is not dissipating for prolonged time periods, say weeks or months. In a preferred embodiment of the present invention, the charge control agent transiently binds to the outer surface of the fibers and therefore the charge dissipates shortly thereafter. This is because polycondensation is not exercised at all such that the chemical interaction between the agent and the polymer is absent, and further due to the low concentration of charge control agent employed. The resulting nonwoven material is therefore, if so desired, substantially charge free.

Suitable charge control agents include, but are not limited to, mono- and poly-cyclic radicals that can bind to the polymer molecule via, for example, —C═C—, ═C—SH— or —CO—NH— groups, including biscationic amides, phenol and uryl sulfide derivatives, metal complex compounds, triphenylmethanes, dimethylmidazole and ethoxytrimethylsians.

Whether or not step 81 is performed, the method continues to step 82 in which the liquefied polymer is dispensed, via an electrospinning process, onto a precipitation electrode, to form the tubular structure thereupon. Generally in the electrospinning process, the liquefied polymer is drawn into a dispensing electrode, and then, subjected to an electric field, dispensed in a direction of the precipitation electrode. Moving with high velocity in the inter-electrode space, jets of liquefied polymer evaporate, thus forming fibers which are collected on the surface of the precipitation electrode. Exemplified system and apparatus suitable for performing the electrospinning process are provided hereinunder.

The procedure by which the polymer is dispensed depends on the application for which the medical device is designed. For example, when the medical device is a stent assembly (see, e.g., FIGS. 1-2), the electrospinning process is employed such as to coat the expandable tubular supporting element by the tubular structure. This can be done in more than one way.

In one embodiment, the supporting element is mounted on a precipitation electrode (e.g., a mandrel), prior to the electrospinning process. In this embodiment, the precipitation electrode function both as a carrier for the supporting element and as a conductive element to which a high voltage is applied to establish the electric field required for activating the electrospinning process. As a consequence, the polymer fibers are projected toward the precipitation electrode and form an outer coat (e.g., coat 16) on the supporting element. This coating covers both the metal wires of the supporting element and gaps between the wires.

In another embodiment, the supporting element serves as a precipitation electrode. In this embodiment, polymer fibers are exclusively attracted to the wires of the supporting element exposing the gaps therebetween. The resultant coated stent assembly therefore has pores which serve for facilitating pharmaceutical agent delivery from the stent assembly into body vasculature.

According to a preferred embodiment of the present invention a liner to the supporting element (e.g., coat 14) is provided as follows. First, the electrospinning process is employed so as to directly coat the mandrel. Once the mandrel is coated, the electrospinning process is temporarily ceased and the supporting element is slipped onto the mandrel and drawn over the coat formed on the mandrel. The outer coat can then provided by resuming the electrospinning process onto the supporting element.

Since the operation for providing the inner coat demands a process cessation for a certain period, a majority of solvent contained in the inner coat may be evaporated. This may lead to a poor adhesion between the components of the stent assembly, once the process is resumed, and might result in the coating stratification following stent graft opening.

The present embodiments successfully address the above-indicated limitation by two optimized techniques. According to one technique, the outer sub-layer of the inner coat and the inner sub-layer of the outer coat are each made by electrospinning with upgraded capacity. A typical upgrading can may range from about 50% to about 100%. This procedure produce a dense adhesion layer made of thicker fibers with markedly increased solvent content. A typical thickness of the adhesion layer ranges between about 20 μm and about 30 μm, which is small compared to the overall diameter of the stent assembly hence does not produce considerable effect on the coats general parameters. According to an alternative technique, the adhesion layer comprises an alternative polymer with lower molecular weight than the major polymer, possessing high elastic properties and reactivity.

Other techniques for improving adhesion between the layers and the supporting element may also be employed. For example, implementation of various adhesives, primers, welding, chemical binding in the solvent fumes can be used. Examples for suitable materials are silanes such as aminoethyaminopropyl-triacytoxysilane and the like.

When the medical device is a vascular prosthesis, a first liquefied polymer is dispensed via electrospinning onto the precipitation electrode to provide a first layer (see, e.g., layer 42 in FIG. 8a) having a predetermined first porosity. A second liquefied polymer is dispensed onto the precipitation electrode to provide a second layer (see, e.g., layer 44 in FIG. 8a) having a predetermined second porosity. The precipitation electrode, which serves for generating the vascular prosthesis thereupon, can be, for example, a rotating mandrel of uniform or varying radius, depending on the size of the vascular prosthesis to be fabricated.

As stated, in preferred embodiments of the invention, the vascular prosthesis may further includes at least one intermediate layer (see e.g., layer 43 in FIG. 8b) interposed between the first and second layers. In such a case, one or more additional liquefied polymers are dispensed onto the precipitation electrode prior to the electrospinning of the second liquefied polymer, to provide the intermediate layer(s) onto the first layer.

The advantage of using the electrospinning method for fabricating the tubular structure of the medical device is flexibility of choosing the polymer types and fibers thickness, thereby providing a final product having the required combination of strength, elastic and other properties as delineated herein. In addition, an alternating sequence of the sub-layers, each made of differently oriented fibers, determines the porosity distribution nature along the tubular structure wall. Still in addition, the electrospinning method has the advantage of allowing the incorporation of various chemical components, such as pharmaceutical agents, to be incorporated in the fibers by mixing the pharmaceutical agents in the liquefied polymers prior to electrospinning.

The method preferably continues to step 83 in which the tubular structure is removed from the precipitation electrode. Preferred techniques for removing the tubular structure from the electrode are provided hereinunder.

The method ends at step 84.

The tubular structure of the medical device can be made of any known biocompatible polymer. In the layers which incorporate pharmaceutical agent, the polymer fibers are preferably a combination of a biodegradable polymer and a biostable polymer.

Suitable biostable polymers which can be used in the present embodiments include, without limitation, polycarbonate based aliphatic polyurethanes, silicon modificated polyurethanes, polydimethylsiloxane and other silicone rubbers, polyester, polyolefins, polymethyl-methacrylate, vinyl halide polymer and copolymers, polyvinyl aromatics, polyvinyl esters, polyamides, polyimides and polyethers.

Suitable biodegradable polymers which can be used in the present embodiments include, without limitation, dextran, dextrin, alginate, hydroxypropyl methylcellulose, hydroxypropyl cellulose, polyvinyl alcohol, poly (L-lactic acid), poly (lactide-co-glycolide), polyethylene glycol, polyethylene oxide, polycaprolactone, polyphosphate ester, poly (hydroxy-butyrate), poly (glycolic acid), poly (DL-lactic acid), poly (amino acid), chitozan, low molecular weight polyvinyl alcohols, high molecular weight polyethylene glycols, hydrophilic polyurethanes, polygalacturonic acid derivatives and cellulose and mixtures thereof.

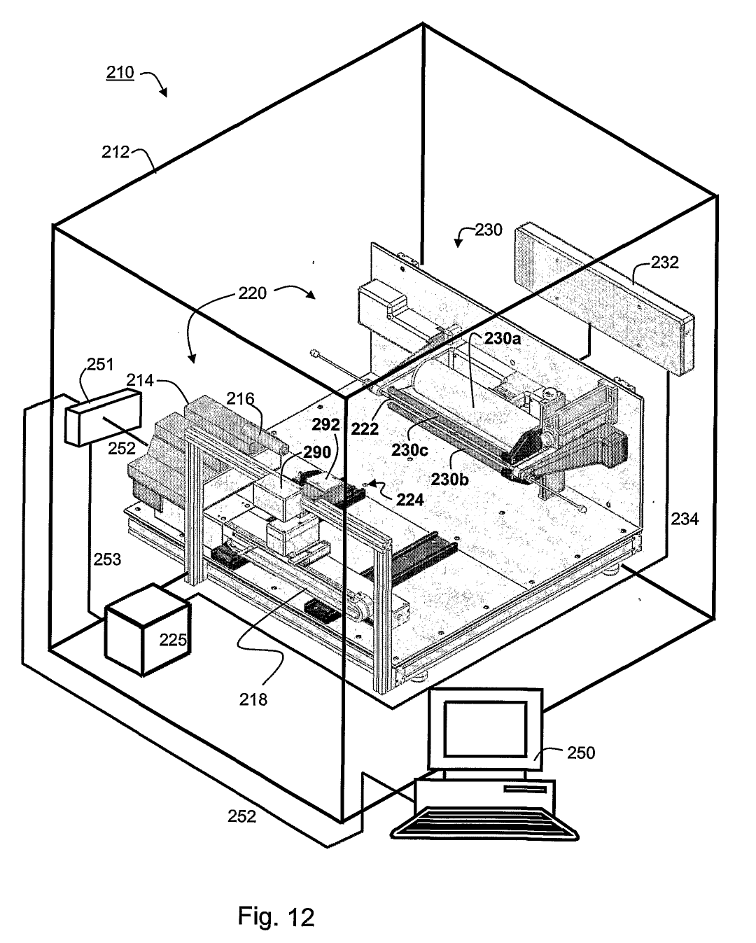

Reference is now made to FIG. 12 which is a schematic illustration of a system 210 for manufacturing an electrospun tubular structure. In its simplest configuration, System 210 comprises an electrospinning system 220 having a precipitation electrode 222, and a dispenser 224, positioned at a predetermined distance from a precipitation electrode 222 and being kept at a first potential relative to precipitation electrode 222.

The potential difference between dispenser 224 and precipitation electrode 222 is preferably from about 10 kV to about 100 kV, typically about 60 kV. The potential difference between dispenser 224 and precipitation electrode 222 generate an electric field therebetween.

Dispenser 224 serves for dispensing a liquefied polymer in the electric field to produce polymer fibers precipitating on electrode 222. Precipitation electrode 222 serves for forming the electrospun tubular structure thereupon. Precipitation electrode 222 is typically manufactured in accordance with the geometrical properties of the final product which is to be fabricated. In various exemplary embodiments of the invention the electrospun tubular structure has a tubular shape and electrode 222 is in a form of a rotating mandrel. Electrode 222 can be made of, for example, stainless steel.

System 210 preferably comprises a subsidiary electrode system 230, which is preferably at a second potential relative to precipitation electrode 222 and configured to shape the aforementioned electric field. Typically, electrode system 230 is connected to source 225 by line 234 and a circuitry 232 which alters (typically reduce) the output voltage of 225 to the desired level. A typical potential difference between electrode 222 and electrode system 230 is from about 10 kV to about 100 kV, typically about 50 kV.

Electrode system 230 may comprise a plurality of electrodes in any arrangement. The size, shape, position and number of electrodes in system 230 is preferably selected so as to maximize the coating precipitation factor, while minimizing the effect of corona discharge in the area of precipitation electrode 222 and/or so as to provide for controlled fiber orientation upon deposition.

In the exemplified configuration shown in FIG. 2, which is not to be considered as limiting, system 230 comprises three cylindrical electrodes, designated 230a, 230b and 230c, where electrode 230a is of larger diameter and is positioned behind precipitation electrode 222, while electrodes 230b and 230c are of smaller diameter and poisoned above and below electrodes electrode 222.

Subsidiary electrode system 230 controls the direction and magnitude of the electric field between precipitation electrode 222 and dispenser 224 and as such, can be used to control the orientation of polymer fibers precipitated on electrode 222. In some embodiments, subsidiary electrode system 230 serves as a supplementary screening electrode. Generally, the use of screening results in decreasing the coating precipitation factor, which is particularly important upon cylindrical precipitation electrodes having at least a section of small radii of curvature.

Electrode shapes which can be used in the present embodiments include, but are not limited to, a plane, a cylinder, a torus a rod, a knife, an arc or a ring.

Specifically, a cylindrical or planar subsidiary electrode enables manufacturing intricate-profile products being at least partially with small (from about 0.025 millimeters to about 5 millimeters) radius of curvature. Such subsidiary electrodes are also useful for achieving random or circumferential alignment of the fibers onto precipitation electrode 222.

The ability to control fiber orientation is important when fabricating electrospun tubular structures in which a high radial strength and elasticity is important. It will be appreciated that a polar oriented structure can generally be obtained also by wet spinning methods, however in wet spinning methods the fibers are thicker than those used by electrospinning by at least an order of magnitude.

Control over fiber orientation is also advantageous when fabricating composite polymer fiber shells which are manufactured by sequential deposition of several different fiber materials.

Subsidiary electrodes of small radius of curvature (e.g., electrodes 230b and 230c), can be used to introduce distortion the electric field in an area adjacent to precipitation electrode 222. For maximum effect the diameter of subsidiary electrode 226 must be considerably smaller than that of precipitation electrode 222, yet large enough to avoid generation of a significant corona discharge.

According to a preferred embodiment of the present invention system 210 the position of subsidiary electrode system 226 can be varied relative to precipitation electrode 222. Such design further facilitates the ability to control the electric field vector (intensity and direction) near electrode 222.

System 210 further comprises a compartment 212, encapsulating electrospinning system 220 and subsidiary electrode system 230. Preferably, but not obligatorily, compartment 212 also encapsulates source 225, and circuitry 232, including the connection lines. Compartment 212 is preferably made of a material being transmissive in the visual range,

Compartment 212 serves for keeping a clean environment therein. According to a preferred embodiment of the present invention the clean environment is of class 1000 (i.e., less than one thousands particles larger than 0.5 microns in each cubic foot of space) or cleaner. More preferably the clean environment is of class 100 (i.e., less than one thousand particles larger than 0.5 microns in each cubic foot of air space).

More specifically, compartment 212 serves as a climate chamber which besides the clean environment, maintains therein predetermined levels of other environmental conditions such as temperature and humidity.

Thus, according to a preferred embodiment of the present invention the temperature with compartment 212 is kept at a predetermined constant level within an accuracy of ±1° C., more preferably ±0.5° C. even more preferably ±0.2° C., so as to control and maintain the desired evaporation rate during the electrospinning process. Maintenance of accurate temperature within compartment 212 is advantageous because the thickness of the produced polymer fibers and the porosity of the electrospun tubular structure, depends, inter alia, on the evaporation rate of solvent from the polymer jets emerge from dispenser 224. Preferred temperatures for the operation are from about 22° C. to about 40° C.

Additionally, the humidity within compartment 212 is maintained at a predetermined level to an accuracy of 5% more preferably 3% even more preferably 1%. Maintenance of accurate temperature within compartment 212 is useful for preventing or reducing formation of volume charge. Preferred humidity level, in relative value (the weight or pressure of moisture relative to the maximal weight or pressure of moisture for a given temperature) is about 40%.

Dispenser 224 and/or precipitation electrode 222 preferably rotates such that a relative rotary motion is established between dispenser 224 and electrode 222. Similarly, Dispenser 224 and/or electrode 222 preferably moves such that a relative linear motion is established between dispenser 224 and electrode 222. In the exemplified configuration shown in FIG. 2, precipitation electrode rotates without performing a linear motion, while dispenser 224 performs a linear motion without performing a rotary motion. However, this need not necessarily be the case, since, for some applications, it may be desired to rotate dispenser 224 about a longitudinal axis 221 of electrode 222 and/or to establish a linear motion of electrode 222 along its longitudinal axis.

When electrode 222 rotates about its axis, the rotation can be established by any mechanism, such as, but not limited to, an electrical motor, an electromagnetic motor, a pneumatic motor, a hydraulic motor, a mechanical gear and the like.

In various exemplary embodiments of the invention system 210 preferably comprises a data processor 250 supplemented by an algorithm for controlling the operation of electrospinning system 220. Data processor 250 can communicate with system 220 directly or through a control unit 251 located within compartment 212. The communication can be via communication line 252 or, more preferably, via wireless communication so as to preserve to clean environment in compartment 212. Preferably, but not obligatorily, processor 250 also communicates (e.g., through control unit 251 and communication line 253) with source 225 for controlling the aforementioned potential differences and for automatically activating and deactivating system 210. According to a preferred embodiment of the present invention processor 250 is configured (e.g., by a suitable computer program) to vary the relative rotary motion and/or relative linear motion between dispenser 224 and electrode 222. For example, when electrode 222 rotates by means of electric motor 240, the power supplied to motor 240, hence the angular velocity of electrode 222 is controlled by processor 250. Similarly, when dispenser 224 moves along convey 254 by means of electric motor 258, the power supplied to motor 258, hence the linear velocity of dispenser 224 is controlled by processor 250.

As will be appreciated by one ordinarily skilled in the art, different angular and/or linear relative velocities can result in different precipitation rates of polymer fibers on electrode 222. Thus, the computerized control on the motions can be used to select the desired precipitation rate, hence also the desired wall thickness of the electrospun tubular structure.

Additionally, processor 250 can signal the mechanism for establishing the linear and/or angular motions of dispenser 224 and/or electrode 222 to change the corresponding velocities, at a given instant or instances of the process. This embodiment is particularly useful when manufacturing multilayer vascular prostheses. Thus, by selecting different motion characteristics of dispenser 224 and/or electrode 222 for different layers, the electrospinning process for each layer is at a different precipitation rate, resulting in a different density of fibers on the formed layer. Since the porosity of the layer depends on the density of fiber, such process can be used for manufacturing multilayer vascular prostheses in which the layers have predetermined and different porosities. Additionally, each layer can have a different wall thickness, which can also be controlled as further detailed above.

The graft of the present embodiments is preferably manufactured as follows.

One or more liquefied polymers are provided and introduced into the electrospinning system. The liquefied polymer(s) can also be mixed with one or more conductivity control agents or charge control agents for improving the interaction of the fibers with the electric field.

The distance between the precipitation electrode and the subsidiary electrodes, the distance between the dispenser and the precipitation electrode, and the angle between the dispenser and the precipitation electrode are adjusted by the adjustments mechanism and recorded into the data processor.

System 210 is sealed by the compartment and the appropriate environmental conditions are established. Parameters, such as, but not limited to, wall thickness, number of layer, angular and linear velocities, temperature, hydrostatic pressure, polymer viscosities, and the like, are recorded into the data processor. Also recorded are the types of polymers.

System 210 is activated and the liquefied polymer is extruded under the action of the hydrostatic pressure through the spinnerets. As soon as meniscus of the extruded liquefied polymer forms, a process of solvent evaporation or cooling starts, which is accompanied by the creation of capsules with a semi-rigid envelope or crust. Because the liquefied polymer possesses a certain degree of electrical conductivity, the capsules become charged by the electric field. Electric forces of repulsion within the capsules lead to a drastic increase in hydrostatic pressure. The semi-rigid envelopes are stretched, and a number of point micro-ruptures are formed on the surface of each envelope leading to spraying of ultra-thin jets of the liquefied polymer from the spinnerets.

Under the effect of a Coulomb force, the jets depart from the dispenser and travel towards the opposite polarity electrode, i.e., the precipitation electrode. Moving with high velocity in the inter-electrode space, the jet cools or solvent therein evaporates, thus forming fibers which are collected on the surface of the precipitation electrode.

Once a first layer is formed, the data processor signals the dispenser to reselect a different liquefied polymer (in embodiments in which different liquefied polymers are used for different layers), and the motion mechanisms to change the rotary and/or linear velocities (in embodiments in which different the layers have different wall thicknesses and/or different porosities). The signaling of the data processor is preferably performed without ceasing the electrospinning process, such that the new layer is formed substantially immediately after the previous layer.

Once all the layers are formed, the compartment is opened and the precipitation electrode, including the tubular structure formed thereupon is disengaged from the system.

According to a preferred embodiment of the present invention the removal of the electrospun product from the precipitation electrode is preferably performed as follows. The precipitation electrode, including the tubular structure, is irradiated by ultrasound radiation. It was found by the inventor of the present invention that ultrasound radiation facilitates the removal of the tubular structure from the electrode. Additionally and more preferably, the precipitation electrode including the tubular structure can also be subjected to at least one substantially abrupt temperature change. The abrupt temperature change can be applied by any suitable heat carrier, including, without limitation, a liquid bath. The removal process can also be controlled by the data processor. Specifically, the data processor can control the duration and level of the applied temperatures and/or the ultrasound radiation.

The process of removal can thus be performed in accordance with various exemplary embodiments of the invention as follows. The precipitation electrode including the tubular structure is immersed in an ultrasonic bath of low temperature (about 0° C.) for a first predetermined period (about 1-10 minutes, more preferably 3-5 minutes). Subsequently, the precipitation electrode including the tubular structure is immersed in another ultrasonic bath of high temperature (from about 40° C. to about 100° C.) for a second predetermined period (about 1-10 minutes, more preferably 3-5 minutes). In experiments performed by the present inventor it was found that the tubular structure can then be removed from the precipitation electrode by an easy manual effort.

If a vascular prosthesis fabricated using the above described procedure requires additional radial strength, radial reinforcing fibers or wires can be added during or following electrospinning. The reinforcing fibers preferably have a diameter from about 300 micrometers to about 500 micrometers.

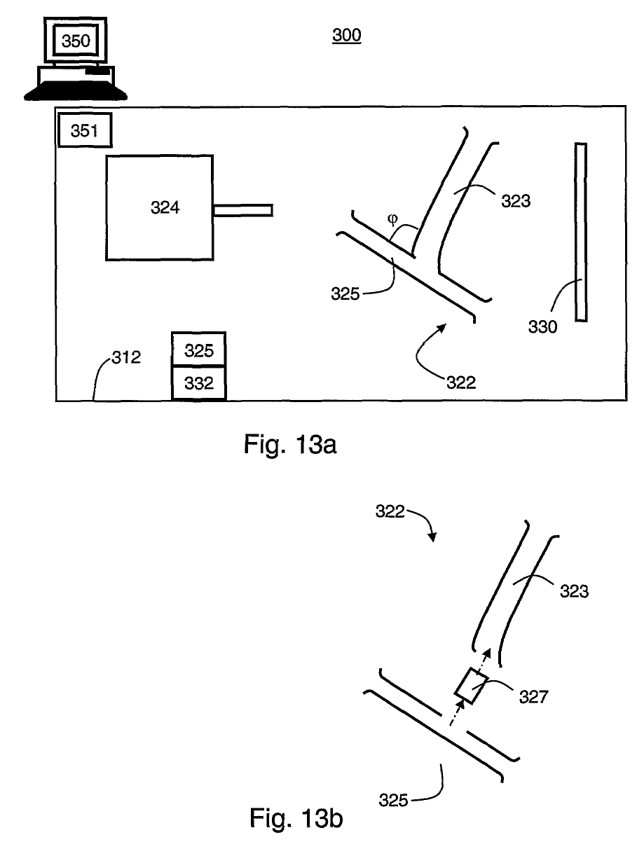

Reference is now made to FIGS. 13a-b, which are schematic illustrations of an apparatus 300 for manufacturing an multiport electrospun structure, according to various exemplary embodiments of the present invention. In its simplest configuration, apparatus 300 comprises a precipitation electrode 322, and a dispenser 324, positioned at a predetermined distance from precipitation electrode 322 and being kept at a first potential relative to precipitation electrode 322.

Precipitation electrode 322 is typically manufactured in accordance with the geometrical properties of the final product which is to be fabricated. In the representative example of FIGS. 13a-b, electrode 322 has a T-shape having arms 323 and 325 (arm 323 terminates on the side of arm 325), to enable manufacturing of multiport structures suitable, e.g., for implantation as arteriovenous shunts, as further detailed hereinabove. Electrode 322 can be made of, for example, stainless steel, or any other electrically conducting material.

The angle φ between arms 323 and 325 is not limited. Preferably, but not obligatorily, φ is below 70°. Electrode 322 is better illustrated in the explosion diagram of FIG. 13b. According to the presently preferred embodiment of the invention arms 323 and 325 of electrode 322 are detachable. For example, arms 323 and 325 can be connected by a removable end-to-side connector 327. The advantage of making arms 323 and 325 detachable is that such configuration facilitates the post manufacturing removal of the final electrospun product from electrode 322.

Alternatively, arms 323 and 325 can have a permanent connection therebetween, such that electrode 322 remains within the lumens of the final multiport structure. This embodiment is particularly when it is desired to manufacture a multiport structure having a mechanical support extending between its ports (such as, for example, support 50 hereinabove). Thus, electrode 322 can serve for post manufacture support of the multiport structure.

Similarly to system 210 above apparatus 300 preferably comprises a subsidiary electrode system 330, which is preferably at a second potential relative to precipitation electrode 322 and configured to shape the aforementioned electric field. Further, apparatus 330 may comprises a compartment 312, which encapsulates dispenser 324, electrode 322 and subsidiary electrode system 330. Preferably, but not obligatorily, compartment 312 also encapsulates the power source 325 and circuitry 332 which supply the power to apparatus 300. The principles of compartment 312 are similar to the principles of compartment 212 as further detailed above.

Dispenser 324 and/or precipitation electrode 322 preferably rotate such that a relative rotary motion is established between dispenser 324 and electrode 322. Similarly, dispenser 324 and/or electrode 322 preferably move such that a relative linear motion is established between dispenser 324 and electrode 322. For example, in one preferred embodiment, precipitation electrode 322 rotates without performing a linear motion, while dispenser 324 performs a linear motion without performing a rotary motion. In another preferred embodiment, dispenser 324 rotates about electrode 322 and electrode 322 performs a linear reciprocal motion. In an additional preferred embodiment, dispenser 324 performs a spiral motion about electrode 322. The relative motion between dispenser 324 and electrode 322 can be established by any mechanism, such as, but not limited to, an electrical motor, an electromagnetic motor, a pneumatic motor, a hydraulic motor, a mechanical gear and the like.

In various exemplary embodiments of the invention apparatus 300 is controlled by a data processor 350 supplemented by an algorithm for controlling apparatus 300. Data processor 350 can communicate with any of the components of apparatus 300 directly or through a control unit 351 located within compartment 312. The communication can be via communication line or, more preferably, via wireless communication so as to preserve to clean environment in compartment 312. Preferably, but not obligatorily, processor 350 also communicates (e.g., through control unit 351) with source 325 and circuitry 332 for controlling the aforementioned potential differences and for automatically activating and deactivating apparatus 300. According to a preferred embodiment of the present invention processor 350 is configured (e.g., by a suitable computer program) to vary the relative rotary motion and/or relative linear motion between dispenser 324 and electrode 322. As will be appreciated by one ordinarily skilled in the art, different angular and/or linear relative velocities can result in different precipitation rates of polymer fibers on electrode 322. Thus, the computerized control on the motions can be used to select the desired precipitation rate, hence also the desired wall thickness of the electrospun structure.

Additionally, processor 350 can signal the mechanism for establishing the linear and/or angular motions of dispenser 324 and/or electrode 322 to change the corresponding velocities, at a given instant or instances of the process. This embodiment is particularly useful when manufacturing multilayer structures. Thus, by selecting different motion characteristics of dispenser 324 and/or electrode 322 for different layers, the electrospinning process for each layer is at a different precipitation rate, resulting in a different density of fibers on the formed layer. Since the porosity of the layer depends on the density of fiber, such process can be used for manufacturing multilayer electrospun structures in which the layers have predetermined and different porosities. Additionally, each layer can have a different wall thickness, which can also be controlled as further detailed above.

A vascular prosthesis fabricated according to the teachings of the present embodiments can be implanted in a subject in need utilizing any well known approach.

For example, the vascular prosthesis is used as an access graft, e.g., an arteriovenous shunt, a pair of openings can be formed in an artery and a vein. Thereafter, the vascular prosthesis can be connected to the pair of openings to allow blood flow from the artery through the vascular prosthesis and into the vein.

When the vascular prosthesis is used for replacing a portion of a blood vessel, the portion of the blood vessel can be excised to create a pair of blood vessel ends. Thereafter the vascular prosthesis can be connected to the pair of blood vessel ends to allow blood flow through the graft.

When the vascular graft is used for bypassing, e.g., an obstructed portion of a blood vessel, a pair of openings can be formed in the blood vessel, upstream and downstream the obstruction. Thereafter, the vascular prosthesis can be connected to the pair of openings to allow blood flow through the vascular graft.

A stent assembly prosthesis fabricated according to the teachings of the present embodiments can be implanted in a subject in need utilizing any well known approach.

For example, the stent assembly can be placed in a constricted blood vessel. Thereafter, the expandable tubular supporting element and tubular structure of the stent assembly can be expanded so as to dilate tissues surrounding the stent assembly in a manner such that flow constriction is substantially eradicated.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A medical device, comprising a tubular structure adapted for being implanted in the vasculature of a mammal, said tubular structure being formed, at least in part, of electrically charged nonwoven polymer fibers.

2. A method of connecting a pair of blood vessels, comprising, providing the medical device of claim 1, forming a pair of holes in the pair of blood vessels, and connecting the medical device to said pair of holes so as to allow blood flow through the medical device, thereby connecting the pair of blood vessels.

3. A method of bypassing an obstructed portion of a blood vessel, comprising, providing the medical device of claim 1, forming a pair of holes in the blood vessel upstream and downstream the obstruction, and connecting the medical device to said pair of holes so as to allow blood flow through the medical device.

4. A method of producing a medical device, comprising electrospinning at least one liquefied polymer onto a precipitation electrode such as to provide a tubular structure formed of electrically charged nonwoven polymer fibers.

5. The method of claim 4, wherein said precipitation electrode comprises a rotating mandrel.

6. The method of claim 4, wherein said precipitation electrode comprises an expandable tubular supporting element.

7. The method of claim 4, wherein said precipitation electrode comprises an expandable tubular supporting element mounted on a rotating mandrel.

8. The method of claim 4, further comprising supplementing said liquefied polymer with a charge control agent, prior to said electrospinning, said charge control agent being selected such that said nonwoven polymer fibers maintain a sufficiently amount of electrical charge for at least T hours.

9. The device of claim 1, further comprising an expandable tubular supporting element.

10. The device of claim 9, wherein said expandable tubular supporting element is coated by said tubular structure.

11. The device of claim 9, wherein said tubular structure serves as a liner for said expandable tubular supporting element.

12. The device of claim 9, wherein said expandable tubular supporting element is embedded within said tubular structure.

13. A method of treating a constricted blood vessel, the method comprising placing the medical device of claim 9 in the constricted blood vessel.

14. The method of claim 13, further comprising expanding said expandable tubular supporting element and said tubular structure so as to dilate tissues surrounding the device in a manner such that flow constriction is substantially eradicated.

15. The device of claim 1, wherein said nonwoven polymer fibers comprise electrospun polymer fibers.

16. The device of claim 1, wherein said tubular structure comprises at least a first layer having a predetermined first porosity and a second layer having a predetermined second porosity.

17. The device of claim 16, wherein said first layer is formed of a first type of nonwoven polymer fibers and said second layer is formed of a second type of nonwoven polymer fibers.

18. The device of claim 1, further comprising a secondary tubular structure of nonwoven polymer fibers, said tubular structure and said secondary tubular structure being in fluid communication via an anastomosis such that said tubular structure terminates at said anastomosis and said secondary tubular structure continues at said anastomosis.

19. The device of claim 1, wherein said tubular structure comprises at least one part positively charged part and at least one negatively charged part.

20. The device of claim 1, wherein said tubular structure has a substantially zero overall net electrical charge.

21. The device of claim 1, wherein said tubular structure has an overall net positive electrical charge of at least 0.001 μC per gram in magnitude.

22. The device of claim 1, wherein said tubular structure has an overall net negative electrical charge of at least 0.001 μC per gram in magnitude.

23. The device of claim 1, wherein said electrically charged nonwoven polymer fibers are capable of discharging at least 90% of the electric charge carried thereby over a predetermined time interval.

24. The device of claim 23, wherein said predetermined time interval is defined from the implantation of the device in the vasculature to about 1 hour following said implantation.

25. The device of claim 23, wherein said predetermined time interval is defined from the implantation of the device in the vasculature to about 12 hours following said implantation.

26. The device of claim 23, wherein said predetermined time interval is defined from the implantation of the device in the vasculature to about 24 hours following said implantation.

27. The device of claim 23, wherein said predetermined time interval is defined from the implantation of the device in the vasculature to about 3 days following said implantation.

28. The device of claim 23, wherein said predetermined time interval is defined from about 3 days following the implantation of the device in the vasculature to about 7 days following said implantation.

29. The device of claim 23, wherein said predetermined time interval is defined from about 7 days following the implantation of the device in the vasculature to about 30 days following said implantation.

30. The device of claim 1, wherein said electrically charged nonwoven polymer fibers are capable of maintaining at least 90% of the electric charge carried thereby over a predetermined period following implantation of the device in the vasculature.

31. The device of claim 30, wherein said predetermined period equals about 3 days.

32. The device of claim 30, wherein said predetermined period equals about 7 days.

33. The device of claim 30, wherein said predetermined period equals about 30 days.

34. The device of claim 1, wherein said tubular structure comprises at least one pharmaceutical agent incorporated therein for delivery of said at least one pharmaceutical agent into said vasculature during or after implantation of the medical device within said vasculature.
